(19) United States
(12) Patent Application Publication
(10) Pub. No.: US 2007/0269503 A1
(43) Pub. Date: Nov. 22, 2007

(54) Combinations of HMG CoA reductase inhibitors and negatively charged phospholipids and uses thereof

(76) Inventors: James Walter Burgess, Nepean (CA); Daniel Sparks, Gatineau (CA)

Correspondence Address: SMART & BIGGAR;P.O. BOX 2999, STATION D, 900-55 METCALFE STREET, OTTAWA, ON, K1P5Y6, US

(21) Appl. No.: 11/434,157

(22) Filed: May 16, 2006

(51) Int. Cl. A61K 31/401; A61K 31/366; A61K 31/22; A61K 9/127

(52) U.S. Cl. 424/450; 514/423; 514/460; 514/548

(57) ABSTRACT

The present invention provides combinations of pharmaceutically active agents (i.e. statins and negatively charged phospholipids) for treating or preventing atherosclerosis or coronary artery disease (CAD) in mammals, and methods therefore. In particular, these combinations and methods find utility for achieving one or more of the following effects in a subject: (1) a reduction in plasma levels of low-density lipoprotein (LDL) cholesterol and very-low-density lipoprotein (VLDL) cholesterol; (2) an increase in plasma levels of high-density lipoprotein (HDL) cholesterol; and (3) a reduction in plasma levels of triglycerides.

### FIELD OF THE INVENTION

The present invention relates to combinations of pharmaceutically active agents and methods for treating or preventing atherosclerosis or coronary artery disease (CAD) in mammals. In particular, these combinations and methods can be used to achieve one or more of the following effects in a subject: (1) a reduction in plasma levels of very-low-density lipoprotein (VLDL) cholesterol and low-density lipoprotein (LDL) cholesterol; (2) an increase in plasma levels of high-density lipoprotein (HDL) cholesterol levels; and (3) a reduction in plasma levels of triglycerides.

### BACKGROUND

Coronary artery disease (CAD) is the leading cause of mortality and morbidity in the USA and most Western countries. As such, CAD is a world-wide health concern.

Most CAD is due to atherosclerosis (a condition characterized by subintimal thickening due to deposition of atheromas) of the large and medium-sized arteries of the heart.

CAD is directly associated with disorders in lipoprotein metabolism (dyslipidemia), including hypercholesterolemia, hypertriglyceridemia, combined hyperlipidemia, and low levels of HDL. Therefore, the medical field has a need for safe and effective treatment options that can achieve target plasma levels of LDL and VLDL cholesterol in patients that suffer from or are at risk of developing atherosclerosis or CAD. Treatments that achieve raising plasma HDL cholesterol and/or lowering plasma triglyceride levels in patients are also known to be beneficial for treating or preventing atherosclerosis or CAD.

### SUMMARY OF THE INVENTION

In one aspect, the present invention provides a combination for use for treating or preventing atherosclerosis or coronary artery disease (CAD) in a mammal comprising: (a) a 3-hydroxy-3-methylglutaryl-CoA (HMG CoA) reductase inhibitor; and (b) a negatively charged phospholipid; wherein the HMG CoA reductase inhibitor and negatively charged phospholipid are present in amounts that render the combination thereof effective for treating or preventing atherosclerosis or coronary artery disease (CAD) in said mammal.

In embodiments, the combination described above is effective for achieving one or more of the following:

(a) a reduction in plasma levels of LDL and VLDL cholesterol in a mammal;

(b) an increase in plasma levels of HDL cholesterol in a mammal; and

(c) a decrease in plasma levels of triglycerides in a mammal.

The combination HMG CoA reductase inhibitor and phospholipid can be administered simultaneously or sequentially. Thus in some embodiments, the HMG CoA reductase inhibitor is in a form suitable for oral administration and the phospholipid is in a form suitable for intravenous administration. In some embodiments, the HMG CoA reductase inhibitor and the phospholipid are both formulated for oral administration. For example, the combination can be in the form of a pharmaceutical composition suitable for oral administration.

In another aspect, the present invention provides a method for treating or preventing atherosclerosis or coronary artery disease (CAD)in a mammal, the method comprising administering to the mammal simultaneously or sequentially a combination comprising: (a) a 3-hydroxy-3-methylglutaryl-CoA (HMG CoA) reductase inhibitor; and (b) a negatively charged phospholipid; wherein the HMG CoA reductase inhibitor and negatively charged phospholipid are present in amounts that render the combination thereof effective for treating or preventing atherosclerosis or coronary artery disease (CAD) in said mammal.

In another aspect, the present invention provides use of a negatively charged phospholipid for enhancing the anti-atherosclerotic effects of an HMG CoA reductase inhibitor.

### DETAILED DESCRIPTION

3-hydroxy-3-methyl-glutaryl coenzyme A (HMG CoA) reductase catalyzes an irreversible step in the cholesterol biosynthetic pathway. Consequently, HMG CoA reductase is an important control site in cholesterol metabolism and it has been found that compounds that HMG CoA reductase inhibitors can be used to reduce plasma LDL and VLDL cholesterol.

Conventional treatments with HMG CoA reductase inhibitors are associated with a number of undesirable side-effects, including: rhabdomyolysis (which can be fatal); shortness of breath; hepatoxicity; gastrointestinal problems; neurological problems, including impaired memory or cognitive function; psychiatric problems; immune system problems (e.g. a lupus-like syndrome); erectile dysfunction; gynecomastia; rash and skin problems; irritability or short temper; heart failure; and increased risk of cancer.

The present invention provides improved compositions and methods for reducing plasma LDL and VLDL levels in a mammal, wherein an HMG CoA reductase inhibitor is administered to the mammal in combination with a negatively charged phospholipid. This combination may reduce the therapeutic dose of HMG CoA reductase inhibitor required to reach target plasma levels of LDL and VLDL cholesterol in the mammal, and may provide additional benefits including raising plasma HDL cholesterol levels and/or lowering plasma triglycerides. Therefore, this combination of active compounds may be more suitable for treating mammals that suffer from complex dyslipidemias than therapies that utilize either active compound alone.

In certain embodiments, the combination of negatively charged phospholipid and HMG CoA reductase inhibitor is in the form of an ester compound, i.e. wherein the HMG CoA reductase inhibitor and phospholipid are linked together by an ester bond. For example, such an ester can be formed by reacting a statin bearing a —C(O)OH group with a phospholipid bearing an alcohol (—OH) group. Such esters may have improved bioavailability and/or efficacy and/or reduced side-effects as compared to the individual active agents.

The mammalian subject can be any mammal, including rabbits, rats and humans.

### A. 3-Hydroxy-3-methyl-glutaryl coenzyme A (HMG CoA) Reductase Inhibitors

In the present context, the term “HMG CoA reductase inhibitor” means a competitive inhibitor of HMG CoA reductase and includes statins. “Statins” are a class of structurally related compounds that act as competitive inhibitors of HMG CoA reductase. Herein, the term “HMG CoA reductase inhibitor” includes pharmaceutically acceptable salts and ester derivatives of such compounds.

Examples of HMG CoA reductase inhibitors suitable for use in the present invention include:
lovastatin and mevinolin (U.S. Pat. No. 4,231,938),
pravastatin sodium (U.S. Pat. No. 4,346,227),
fluvastatin (U.S. Pat. Nos. 4,739,073 and 5,354,772),
atorvastatin (U.S. Pat. No. 5,273,995),
itavastatin (European Patent No. 0304063),
mevastatin (U.S. Pat. No. 3,983,140),
rosuvastatin, velostatin, and synvinolin, and simvastatin (U.S. Pat. Nos. 4,448,784 and 4,450,171], and their pharmaceutically acceptable salts and ester derivatives.

The foregoing compounds may be marketed under different names. For example, rosuvastatin is currently being marketed in the USA under the brand name “Crestor®” (AstraZeneca) and atorvastatin is currently being marketed in the USA under the brand name “Lipitor®” (Pfizer Inc.).

Preferred HMG CoA reductase inhibitors for use in the present invention include rosuvastatin calcium and atorvastatin calcium.

### B. Phospholipids

Herein, the term “phospholipid” includes pharmaceutically acceptable salts and ester derivatives of such compounds. The term “negatively charged phospholipid” refers to phospholipids that carry a net negative charge at physiological pH levels, i.e. over the range of about pH 7.3 to 7.5.

We have previously shown that administration of phosphatidylinositol (PI) to a mammal can reduce plasma LDL and VLDL cholesterol levels (U.S. Pat. No. 6,828,306 incorporated herein by reference). Administration of PI to a mammal can also raise plasma levels of HDL cholesterol (U.S. Pat. No. 6,828,306; Burgess, J. W., et al. (2005) J. Lipid Res. 46, 350-355; Burgess, J. W., et al. (2003) J. Lipid Res. 44, 1355-1363) and stimulate hepatic lipase, i.e. to remove triglycerides form the blood (U.S. Pat. No. 6,828,306).

PI can be metabolized in the body to provide a variety metabolites, including singly or multiply phosphorylated phosphatidyl inositols (e.g. PIP, PIP₂, PIP₃, PI(3,5)P₂ and PI(3,4,5)P₃) and the classical intracellular messengers inositol-3,4,5-triphosphate (IP₃) and diacylglycerol (DAG). One or more of these metabolites may be involved in the above-described PI-induced modulation of plasma lipids. For example, it is known that the liver receptor homolog 1(LRH-1), which is a transcriptional activator of the apolipoprotein A-I (apoA-I) gene, protein binds a molecule of phosphorylated phosphatidyl inositol and this ligand binding interaction is required for maximal LRH-1 activity (Krylova, I. N. et al. (2005) Cell. vol. 120, 343-355).

Phosphatidylserine (PS), another negatively charged phospholipid, also modulates cholesterol metabolism (U.S. Pat. No. 6,828,306).

Phosphatidic acid (PA), another negatively charged phospholipid, has also been shown modulate cholesterol metabolism in rats. PA can decrease plasma triglycerides by about 40% and can increase HDL cholesterol by about 2-fold in normocholesterolemic male Sprague-Dawley rats (Patent Abstracts of Japan, publication number 09-000206). The effective administered dose in these experiments represented 5.5% by weight of rat chow, which is equivalent to approximately 4 g/Kg body weight. This study did not report a significant reduction in plasma LDL or VLDL cholesterol, but the study was carried out in rats which are known in the art to be a poor animal model for studying metabolism of LDL and VLDL cholesterol.

Therefore, based on reports in the literature regarding the actions of several negatively charged phospholipids (PI, PA, and PS), we think that negatively charged phospholipids may constitute a class of compounds that have utility for modulating cholesterol metabolism. More particularly, these compounds may be useful as agents for lowering plasma levels of LDL and VLDL cholesterol, raising plasma levels of HDL cholesterol and triglycerides. Negatively charged phospholipids are structurally distinct from HMG CoA reductase inhibitors (e.g. statins) and so it is believed that they lower LDL and VLDL cholesterol by a mechanism other than competitive inhibition of HMG CoA reductase.

Accordingly, the present invention can be practised using negatively charged phospholipids having the following formula I:

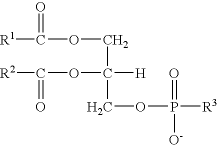

wherein

each of R¹ and R² is independently a C₁₋₂₃ hydrocarbyl group;

R³ is O⁻ or a C₁₋₁₀ hydrocarbyloxy group; and

R¹ and R² and R³ are chosen such that the phospholipid has a net charge of −1, −2 or −3;

and pharmaceutically acceptable salts, protonated forms, and ester derivatives thereof.

Preferred phospholipids have a net charge of −1 or −2 in ionized form.

Suitable values for R¹ and R² include alkyl, alkenyl, and alkadienyl groups having between 3 and 23 carbon atoms, e.g. between 13 to 23 carbon atoms, or between 15 to 19 carbon atoms. For the most part, it is contemplated that R¹ and R² will be unbranched and unsubstituted. However, branching may be acceptable, provided that such branching does not interfere with the utility of the phospholipid. The presence of heteroatoms or subsituents may be acceptable, provided that such heteroatoms and substituents do not interfere with utility of the phospholipid. Suitable substituents include hydroxy, alkoxy (of an alkyl group), and mercapto (of an alkyl group). Suitable heteroatoms include O and S.

As shown in formula I, R¹ and R² are covalently bound to —C(O)— to form an acyl radical. Examples of suitable values for the corresponding R¹—C(O)— and R²—C(O)— acyl radicals include but are not limited to:

tetradecanoyl [myristoyl (14:0)],

9-cis-tetradecenoyl [myristoleoyl (14:1)],

hexadecanoyl [palmitoyl (16:0)],

9-cis-hexadecenoyl [palmitoleoyl (16:1)],

9-trans-hexadecenoyl(palmitoleoyl 16:1),

octadecanoyl [stearoyl (18:0)],

6-cis-octadecenoyl [petroselinoyl (18:1)],

9-cis-octadecenoyl [oleoyl (18:1)],

9-trans-octadecadienoyl [elaidoyl(18:1)],

9-cis-12-cis-octadecadienoyl [linoleoyl (18:2)],

9-cis-12-cis-15-cis-octadecatrienoyl [linolenoyl (18:3)],

11-cis-eicosenoyl [eicosenoyl (20:1)],

5-cis-8-cis-11-cis-14-cis-eicosatetraenoyl [arachidonoyl (20:4)],

13-cis-docosenoyl [erucoyl (22:1)], and

15-cis-tetracosenoyl [nervonyl (24:1)].

Suitable values for R³ when it is a C₁₋₁₀ hydrocarbyloxy group include C₁₋₁₀ alkoxy and C₅₋₆ cycloalkyloxy. R³ can be branched or unbranched and can contain substituents or heteroatoms that do not interfere with the intended utility of the phospholipid. Suitable substituents include hydroxy, alkoxy (of an alkyl group), and mercapto (of an alkyl group). Suitable heteroatoms include O and S.

Accordingly, suitable values for R³ include oxy radicals of: serine, inositol, glycerol, and sugars (such as glucose, fructose etc.).

Phospholipids of formula I can be naturally-occurring phospholipids that have been obtained from a natural source or prepared using standard chemistry. Non-naturally occurring phospholipids of formula I can also be prepared by chemical synthesis, and these phospholipids may be useful, or even preferred in some cases, for practicing the present invention.

Phospholipids of formula I can be partially hydrolyzed (i.e. to remove R¹ and/or R²) to obtain the corresponding lyso-phospholipids, which may also be useful for practicing the present invention. Therefore, in the present context, the term “phospholipid” includes both phospholipids of formula I and their corresponding lyso-phospholipids.

Phospholipids for use in the present invention may be purified or isolated or substantially pure. A compound is “substantially pure” when it is separated from the components that naturally accompany it. Typically, a compound is substantially pure when it is at least 60%, more generally 75% or over 90%, by weight, of the total material in a sample. A substantially pure phospholipid can be obtained, for example, by extraction from a natural source or by chemical synthesis. Thus, for example, a phospholipid that is chemically synthesised will generally be substantially free from its naturally associated components. Purity can be measured using any appropriate method such as column chromatography, gel electrophoresis, HPLC, etc. However, it is not essential for a negatively charged phospholipid to be purified prior to use in the present invention, provided that the phospholipid is not associated with components that interfere substantially with its utility. The skilled person will appreciate that a natural source or partially-purified source of a negatively charged phospholipid may be used in the invention, and that the negatively charged phospholipid component may constitute a small percentage (for example 10-20%, but preferably at least 30%, 40%, 50% or more) of the total material obtained from such a source.

### C: Intestinal Absorption Enhancers

Peroral delivery of is one of the greatest challenges in biopharmaceutical research. The oral route is the preferred route of administration, but many promising drugs present low bioavailability when administered orally. As a result, intestinal absorption enhancement is an active area of research (for a recent review, see: Cano-Cebrian et al. Current Drug delivery, 2005, Volume 2, pp. 9-22).

Intestinal absorption enhancers (IAEs) are compounds that may be used to improve uptake (bioavailability) of orally administered drugs. An enormous variety of compounds have been tested as potential IAEs, including: calcium chelators; medium-chain fatty acids; medium-chain glycerides; steroidal detergents (e.g. bile acids); acylcarnitines; and chitosans and other mucoadhesive poymers (see Cano-Cebrian et al. (2005), supra).

We have found that, using conventional formulations, large oral doses of PI (e.g. about 5.6 g/day) are required to achieve clinically significant increases in plasma HDL cholesterol (˜20%) and its major protein component, apoA-I (˜10%). The efficacy of orally administered phosphatidyl-inositol(PI) for raising plasma HDL cholesterol and apoA-I levels can be improved considerably (e.g. 2- to 8-fold) by combining PI with an intestinal absorption enhancer (IAE) (U.S. patent application Ser. No. 60/684212, herein incorporated by reference).

Suitable IAEs for practising the present invention increase the efficacy of a selected negatively charged phospholipid (measured in terms of a reduction in plasma LDL and VLDL cholesterol, and optionally an increase plasma HDL cholesterol levels) by at least 10% and preferably 20%, 30%, 40%, 50%, 60%, 70%, 80%, 90%, 100%, 200%, 300%, 400% or more.

IAEs may exhibit toxicity and/or side-effects. Accordingly, they should be used in amounts and circumstances where the therapeutic benefit to the patient outweighs any undesirable side-effects.

The following classes of compounds may be useful as IAEs: bile acids; surfactants; medium chain fatty acids; and pharmaceutically acceptable salts thereof. Certain proteolytic enzymes, such as bromelain, may also be useful as IAEs (see for example: Guggi, D. and Bernkop-Schnurch, A. (2005) Int. J. Pharm. 288, 141-150).

The following bile acids may be useful as IAEs:
deoxycholate (Uchiyama, T., et al. (1999) J. Pharm. Pharmacol. 51, 1241-1250; Sakai, M., et al. (1998) J. Pharm. Pharmacol. 50, 1101-1108);
taurocholate (Yamamoto, A., et al. (1996) J. Pharm. Pharmacol. 48, 1285-1289);
chenodeoxycholate (Fricker, G., et al. (1996) Br. J. Pharmacol. 117, 217-223);
glycocholate (Lindhardt, K. and Bechgaard, E. (2003) Int. J. Pharm. 252, 181-186; Bechgaard, E., et al. (1999) Int. J. Pharm. 182, 1-5);
cholate, ursodeoxycholate, (Fricker, G., et al. (1996) Br. J. Pharmacol. 117, 217-223); and
taurodihydrofusidate (Aungst, B. J. (1993) J. Pharm Sci. 82, 979-987.

Surfactants may be used as IAES, provided that they are biocompatible. Specific examples of suitable surfactants include: Tween 20 and sodium lauryl sulfate (SLS). SLS is included in the FDA Inactive Ingredient Guide and is employed in a wide range of pharmaceutical formulations as an anionic surfactant or emulsifying agent. See: Swenson, E. S., et al. (1994) Pharm. Res. 11, 1132-1142; and Anderberg, E. K., et al. (1992) J. Pharm. Sci. 81, 879-887.

Suitable medium chain fatty acids (MCFAs) contain between 8 and 12 carbon atoms. Specific examples of MCFAs include: caprylate (CH₃—(CH₂)₆—COOH); caprate (CH₃—(CH₂)₈—COOH); and laurate (CH₃—(CH₂)₁₀—COOH). In many cases, the sodium salts of such MCFAs will be used, namely: sodium caprylate; sodium laurate (Yata, T., et al. (2001) J. Pharm. Sci. 90, 1456-1465; Miyake, M., et al. (2003) J. Pharm. Sci. 92, 911-921); and sodium caprate (see Cano-Cebrian et al. (2005) supra).

### D. Therapeutic Formulations

The present combination of HMG CoA reductase inhibitor (HRI) and negatively charged phospholipid (PL) may be formulated as a single pharmaceutical composition having both active agents, or as separate pharmaceutical compositions designed for simultaneous or sequential co-administration.

In many cases, the inventive compositions will be formulated as pills, capsules or tablets suitable for oral administration. However, the inventive compositions can also be formulated for other modes of administration, including intravenous and rectal administration.

A “therapeutically effective amount” refers to an amount effective, at dosages and for periods of time necessary, to achieve the desired therapeutic result, such as a clinically significant reduction in plasma LDL and VLDL cholesterol levels and in turn a reduction or reversal in CAD-related disease progression. Herein, a “therapeutically effective amount” includes amounts effective, at dosages and for periods of time necessary, to achieve a prophylactic result, such as preventing or inhibiting the rate of CAD-related disease onset or progression. The therapeutically effective amount of the present combination of HRI/PL may vary according to factors such as the disease state, age, sex, and weight of the individual, and the ability of the particular combination of HRI/PL to elicit a desired response in the individual. Dosage regimens may be adjusted to provide the optimum therapeutic response. A therapeutically effective amount is also one in which any toxic or detrimental effects of the compound are outweighed by the therapeutically beneficial effects.

For any particular subject, specific dosage regimens may be adjusted over time according to the individual need and the professional judgement of the person administering or supervising the administration of the compositions.

Typically, a unit dose may comprise, per kg of body weight of the mammal being treated: (a) between about 0.01 mg to about 30 mg of HRI and (b) between about 0.01 mg to about 140 mg of phospholipid (PL). For example, the combination may comprise between about 0.5 mg to about 3 mg of HRI and between about 0.1 mg to about 14 mg of PL (e.g. between about 0.2 mg to about 4 mg of PL).

Effective doses may vary according to a number of factors (see above), and dosage regimens may be adjusted to provide the optimum therapeutic or prophylactic response. In particular, where an intestinal absorption enhancer is used to enhance bioavailibility of the PL, the amount of PL can be decreased, for example 2- to 8-fold.

Pharmaceutical compositions comprising the present combination of HRI/PL may comprise a pharmacologically acceptable excipient or carrier.

As used herein “pharmaceutically acceptable carrier” or “excipient” includes any and all solvents, dispersion media, coatings, antibacterial and antifungal agents, isotonic and absorption delaying agents, and the like that are physiologically compatible and suitable for oral administration. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Moreover, the present combination of HRI/PL can be administered in a time release formulation, for example in a composition which includes a slow release polymer. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are patented or generally known to those skilled in the art.

Specific examples of orally administrable pharmaceutical compositions include dry-filled capsules consisting of gelatin, and also soft sealed capsules consisting of gelatin and a plasticizer, such as glycerol or sorbitol. The dry-filled capsules may contain the active ingredient in the form of granules, for example in admixture with fillers, such as lactose, binders, such as starches, and/or glidants, such as talc or magnesium stearate, and optionally stabilisers. In soft capsules, the active ingredient is preferably dissolved or suspended in suitable liquids, such as fatty oils, paraffin oil or liquid polyethylene glycols, to which stabilisers may also be added.

The invention provides corresponding methods of medical treatment, in which a therapeutically effective amount of the present combination of HRI/PL is in a pharmacologically acceptable formulation for administering orally (or rectally or buccally) to a mammal subject in need thereof. It is convenient for the present combination of HRI/PL to be administered simultaneously, e.g. with HRI either in admixture with PL in a combination pill or formulated as separate pills that are to be taken at the same time. However, this is not essential and it may be possible to administer the combination of HRI and PL separately, for example as separate pills which may have similar or different dosage regimens.

All publications and patent applications cited in this specification are herein incorporated by reference as if each individual publication or patent application were specifically and individually indicated to be incorporated by reference.

The citation of any publication is for its disclosure prior to the filing date and should not be construed as an admission that the present invention is not entitled to antedate such publication by virtue of prior invention.

### EXAMPLES

### Example 1

### Effects of Atorvastatin/Phosphatidylinositol Combination in HepG2 Cells

HepG2 cells respond to statins with an inhibition of HMGCA reductase and reductions in the synthesis of cholesterol ((Bonn et al.; Funatsu et al.; Gerber, Ryan, and Clark; Maejima et al.; Scharnagl et al.; Wilcox, Barrett, and Huff), increased LDL receptor mRNA (Scharnagl et al.), decreased apoB and (Wilcox, Barrett, and Huff; Funatsu et al.) and triglyceride secretion (Funatsu et al.). HepG2 cells also respond to PI (Burgess et al.) with decreased production of cholesterol and cholesterol esters and with increased secretion of apoA-I.

Accordingly, candidate combinations of statins and phospholipids can be tested in HepG2 cells to assess their effects on cholesterol synthesis, LDL receptor levels, apolipoprotein secretion and LDL production.

For example, atorvastatin and phosphatidylinositol (PI) can be administered to HepG2 cells singly and in combination over a range of concentration ranges, to find concentrations of each inhibitor suitable for observing additive or synergistic effects. Typically, atorvastatin and PI are administered to HepG2 cells in amounts that, if administered singly, would typically produce less than 50% maximal response. In HepG2 cells it is reported that atorvastatin inhibits cholesterol synthesis by 45-65% when used at concentrations between 0.01 to 10 μM (Mohammadi et al.). Therefore, a suitable atorvastatin concentration range may include concentrations of 0.01 μM and less. PI at a concentration of 11.7 μM inhibits cholesterol synthesis by 50% in HepG2 cells (Burgess et al.). Therefore, a suitable PI concentration range may include 11.7 μM and less.

Briefly, HepG2 cells are seeded in 12-well-plates and grown to about 70% confluence. The monolayers of HePG2 cells are pulse-labeled with [¹⁴C]-acetate at a final concentration of 35 mM (2 mCi/L medium) and incubated for 5 h with drugs alone or in combination and at the concentrations described above. After incubation, the cells are washed three times with 1 mL of 150 mM NaCl and suspended in 1 mL of n-hexane:isopropanol (3:2 by volume). After adding 0.25 μCi of [1,2-³H]-cholesterol as an internal standard, each monolayer is extracted for 30 min. The resulting extracts are transferred to glass tubes and centrifuged at 3300 g for 20 min. The lipid phase is removed, evaporated to dryness under a stream of nitrogen, and resuspended in 1 mL of chloroform:methanol (2:1 by volume). The cell pellet is dissolved in 1 mL of 0.2 M NaOH and used for the determination of cell protein. The lipid extracts are subjected to thin-layer chromatography on silica gel plates (Merck, West Point, Pa., USA). The plates are developed with a solvent of hexane:isopropanol:formic acid (80:30:2 by volume). The spots containing triacylglycerides, nonesterified, and esterified cholesterol are visualized by iodine vapour, cut out, and counted in a scintillation counter. The data is expressed as nmol of [¹⁴C]-acetate incorporated per hour and per mg of total cell protein.

### Example 2

### Effects of Atorvastatin/Phosphatidylinositol Combination Treatment in New Zealand White (NZW) rabbits

NZW rabbits respond to statins with reductions in plasma total cholesterol (Alegret et al.; Auerbach et al.; Bocan et al.; Verd et al.; Zhao and Wu), lipoprotein associated cholesterol (Bocan et al.) and triglycerides (Verd et al.). Statins that have been tested in rabbits include atorvastatin, fluvastatin, pravastatin and simvastatin. At doses of 10-20 mg/day in 2 kg rabbits these statins reduce plasma cholesterol levels by approximately 60% (Jorge et al.). Therefore, suitable doses of statins for testing for additive or synergistic effect may be on the order of less than the 10-20 mg/day dose.

The effects of orally administered PI on plasma total cholesterol, lipoprotein cholesterol and triglycerides in rabbits have not yet been properly assessed. To determine the effect of PI of plasma cholesterol in rabbits, NZW rabbits are fed diets with varying amounts of cholesterol ranging from 0.15% to 0.5% of the total daily intake, to elevate plasma cholesterol to different levels (Kolodgie et al.). PI can be administered daily with chow at a dose of 120 mg/kg body weight.

To study synergistic effects of statins and PI, male NZW rabbits weighing approximately 2 kg, are divided into the following 4 groups (n=about 7): 1) hypercholesterolemic control (no drugs administered); 2) atorvastatin; 3) PI; 4) atorvastatin and PI. The animals will be separated in individual cages and fed a standard preparation of Purina brand food, enriched with cholesterol for 4 months. Atorvastatin and PI are administered to the respective groups with chow.

Blood samples are obtained at 2 week intervals until the end of the study. The blood samples are analyzed for total cholesterol as well as lipoprotein-associated cholesterol, apolipoprotein and triglyceride levels as is standard in the literature.

### Example 3

### Effects of Atorvastatin/Phosphatidylinositol Combination Treatment in Humans

The combination of statin and PI can also be tested clinically, typically orally, in humans substantially as described above (in Example 2) for rabbits. Specifically, human subjects are assigned to one of four groups: (1) control (no atorvastatin or PI to be administered); (2) atorvastatin only; (3) PI only; or (4) combination of atrovastatin/PI. Atorvastatin and PI are administered in amounts that, if administered singly, would typically produce less than 50% maximal response. Blood samples are obtained at 2 week intervals until the end of the study. The blood samples are analyzed for total cholesterol as well as lipoprotein-associated cholesterol, apolipoprotein and triglyceride levels in accordance with standard procedures in the art.

### Example 4

### Effects of Various Statin/Phospholipid Combination Treatments in HepG2 Cells, Rabbits and Humans

The experiments described above in Examples 1 to 3 can be used to test the synergistic effects of various combinations of statins and negatively charged phospholipids. That is, atorvastatin can be substituted with another statin, and/or PI can be substituted with another negatively charged phospholipid as described herein. In order to observe synergistic effects, the subject compounds are administered in amounts that, when administered singly, would produce typically less than 50% maximal response.

### REFERENCE LIST

Alegret, M., et al. “Effect of hypolipidemic drugs on key enzyme activities related to lipid metabolism in normolipidemic rabbits.” Eur.J.Pharmacol. 347.2-3 (1998): 283-91.
Auerbach, B. J., et al. “Comparative effects of HMG-CoA reductase inhibitors on apo B production in the casein-fed rabbit: atorvastatin versus lovastatin.” Atherosclerosis 115.2 (1995): 173-80.
Bocan, T. M., et al. “HMG-CoA reductase and ACAT inhibitors act synergistically to lower plasma cholesterol and limit atherosclerotic lesion development in the cholesterol-fed rabbit.” Atherosclerosis 139.1 (1998): 21-30.
Bonn, V., et al. “Simvastatin, an HMG-CoA reductase inhibitor, induces the synthesis and secretion of apolipoprotein AI in HepG2 cells and primary hamster hepatocytes.” Atherosclerosis 163.1 (2002): 59-68.
Burgess, J. W., et al. “Phosphatidylinositol promotes cholesterol transport and excretion.” J.Lipid Res. 44.7 (2003): 1355-63.
Funatsu, T., et al. “Prolonged inhibition of cholesterol synthesis by atorvastatin inhibits apo B-100 and triglyceride secretion from HepG2 cells.” Atherosclerosis 157.1 (2001): 107-15.
Gerber, R., J. D. Ryan, and D. S. Clark. “Cell-based screen of HMG-CoA reductase inhibitors and expression regulators using LC-MS.” Anal.Biochem. 329.1 (2004): 28-34.
Jorge, P. A., et al. “[Effects of atorvastatin, fluvastatin, pravastatin, and simvastatin on endothelial function, lipid peroxidation, and aortic atherosclerosis in hypercholesterolemic rabbits].” Arg Bras.Cardiol. 84.4 (2005): 314-19.
Kolodgie, F. D., et al. “Hypercholesterolemia in the rabbit induced by feeding graded amounts of low-level cholesterol. Methodological considerations regarding individual variability in response to dietary cholesterol and development of lesion type.” Arterioscler. Thromb. Vasc. Biol. 16.12 (1996): 1454-64.
Krylova, I. N. et al. (2005) Cell. vol. 120, 343-355.
Maejima, T., et al. “Effect of pitavastatin on apolipoprotein A-I production in HepG2 cell.” Biochem. Biophys. Res. Commun. 324.2 (2004): 835-39.
Mohammadi, A., et al. “Effects of atorvastatin on the intracellular stability and secretion of apolipoprotein B in HepG2 cells.” Arterioscler. Thromb. Vasc. Biol. 18.5 (1998): 783-93.
Scharnagl, H., et al. “Effect of atorvastatin, simvastatin, and lovastatin on the metabolism of cholesterol and triacylglycerides in HepG2 cells.” Biochem. Pharmacol. 62.11 (2001): 1545-55.
Verd, J. C., et al. “Different effect of simvastatin and atorvastatin on key enzymes involved in VLDL synthesis and catabolism in high fat/cholesterol fed rabbits.” Br. J. Pharmacol. 127.6 (1999): 1479-85.
Wilcox, L. J., P. H. Barrett, and M. W. Huff. “Differential regulation of apolipoprotein B secretion from HepG2 cells by two HMG-CoA reductase inhibitors, atorvastatin and simvastatin.” J. Lipid Res. 40.6 (1999): 1078-89.
Zhao, S. P. and Z. H. Wu. “Atorvastatin reduces serum leptin concentration in hypercholesterolemic rabbits.” Clin. Chim. Acta 360.1-2 (2005): 133-40.

1. A combination for use for treating or preventing atherosclerosis or coronary artery disease (CAD) in a mammal comprising:
(a) a 3-hydroxy-3-methylglutaryl-CoA (HMG CoA) reductase inhibitor; and
(b) a negatively charged phospholipid;
wherein the HMG CoA reductase inhibitor and negatively charged phospholipid are present in amounts that render the combination thereof effective for treating or preventing atherosclerosis or coronary artery disease (CAD) in said mammal.

2. The combination of claim 1, wherein the negatively charged phospholipid is in a form suitable for intravenous administration and the HMG CoA reductase inhibitor is in a form suitable for oral administration.

3. The combination of claim 1, wherein the negatively charged phospholipid and the HMG CoA reductase inhibitor are in the form of a pharmaceutical composition.

4. The combination of claim 3, wherein said pharmaceutical composition is suitable for oral administration.

5. The combination of claim 1 wherein the HMG CoA reductase inhibitor and negatively charged phospholipid are present in amounts that render the combination thereof effective for reducing plasma levels of LDL and/or VLDL cholesterol in a mammal.

6. The combination of claim 1 wherein the HMG CoA reductase inhibitor and negatively charged phospholipid are present in amounts that render the combination thereof effective for increasing the plasma levels of HDL cholesterol in the mammal.

7. The combination of claim 1, wherein the HMG CoA reductase inhibitor and negatively charged phospholipid are present in amounts that render the combination thereof effective for reducing the plasma levels of triglyceride in the mammal.

8. The combination of claim 1 wherein the mammal is a human.

9. The combination of claim 1 wherein the HMG CoA reductase inhibitor is selected from the group consisting of: lovastatin; mevinolin; pravastatin sodium; fluvastatin; atorvastatin; itavastatin; mevastatin; rosuvastatin; velostatin; synvinolin; and simvastatin.

10. The combination of claim 1 wherein the HMG CoA reductase inhibitor is the calcium salt of atorvastatin.

11. The combination of claim 1 wherein the HMG CoA reductase inhibitor is the calcium salt of rosuvastatin.

12. The combination of claim 1 wherein the negatively charged phospholipid is selected from the group consisting of: phosphatidylinositol (PI); lyso-phosphatidylinositol (lyso-PI); phosphorylated phosphatidylinositol (PPI); phosphatidylserine (PS); phosphatidic acid (PA); and phoshatidylglycerol (PG).

13. The combination of claim 1 wherein the negatively charged phospholipid is PI.

14. The combination of claim 3, wherein the pharmaceutical composition further comprises an intestinal absorption enhancer selected from the group consisting of: a bile acid or salt thereof; a surfactant; and a medium chain fatty acid or salt thereof.

15. The combination of claim 14, wherein said intestinal absorption enhancer is sodium lauryl sulfate.

16. A method for treating or preventing atherosclerosis or coronary artery disease (CAD)in a mammal, the method comprising administering to the mammal simultaneously or sequentially a combination comprising:
(a) a 3-hydroxy-3-methylglutaryl-CoA (HMG CoA) reductase inhibitor; and
(b) a negatively charged phospholipid;
wherein the HMG CoA reductase inhibitor and negatively charged phospholipid are present in amounts that render the combination thereof effective for treating or preventing atherosclerosis or coronary artery disease (CAD)in said mammal.

17. The method of claim 16, wherein the (HMG CoA) reductase inhibitor and the negatively charged phospholipid are administered orally.

18. The method of claim 16, wherein the (HMG CoA) reductase inhibitor is administered orally and the negatively charged phospholipid is administered intravenously.

19. A method of claim 16 wherein said combination is effective for reducing plasma levels of LDL and VLDL cholesterol in the mammal.

20. The method of claim 16 wherein said combination is effective for achieving an increase in plasma levels of HDL cholesterol in the mammal.

21. The method of claim 16 wherein said combination is effective for achieving a reduction in plasma levels of triglyceride in the mammal.

22. The method of claim 16 wherein the mammal is a human.

23. The method of claim 16 wherein the HMG CoA reductase inhibitor is selected from the group consisting of: lovastatin; mevinolin; pravastatin sodium; fluvastatin; atorvastatin; itavastatin; mevastatin; rosuvastatin; velostatin; synvinolin; and simvastatin.

24. The method of claim 16 wherein the HMG CoA reductase inhibitor is the calcium salt of atorvastatin.

25. The method of claim 16 wherein the HMG CoA reductase inhibitor is the calcium salt of rosuvastatin.

26. The method of claim 16 wherein the negatively charged phospholipid is selected from the group consisting of: phosphatidylinositol (PI); lyso-phosphatidylinositol (lyso-PI); phosphorylated phosphatidylinositol (PPI); phosphatidylserine (PS); phosphatidic acid (PA); and phoshatidylglycerol (PG).

27. The method of claim 16 wherein the negatively charged phospholipid is PI.

28. The method of claim 16 wherein said combination is formulated for oral administration.

29. The method of claim 27, wherein said combination is co-administered with an intestinal absorption enhancer selected from the group consisting of: a bile acid or salt thereof; a surfactant; and a medium chain fatty acid or salt thereof.

30. The method of claim 29, wherein said intestinal absorption enhancer is sodium lauryl sulfate.
